(19) United States
(12) Patent Application Publication
(10) Pub. No.: US 2019/0285632 A1
(43) Pub. Date: Sep. 19, 2019

(54) METABALOMICS AND VIRAL DIAGNOSTICS SUITE

(71) Applicant: Excision Biotherapeutics, Inc., Andover, NJ (US)

(72) Inventor: Thomas Malcolm, Andover, NJ (US)

(73) Assignee: Excision Biotherapeutics, Inc., Andover, NJ (US)

(21) Appl. No.: 16/300,654

(22) PCT Filed: May 24, 2017
(86) PCT No.: PCT/US17/34164

§ 371 Date: Nov. 12, 2018

Related U.S. Application Data
(60) Provisional application No. 62/340,624, filed on May 24, 2016.

(51) Int. Cl. G01N 33/569

(52) U.S. Cl. CPC G01N 33/56983; G01N 2800/52; G01N 2800/56; G01N 2800/26

(57) ABSTRACT

A diagnostic panel including a test for detecting at least one biomarker that indicates the presence of a virus. A kit including the diagnostic panel, instructions for use, materials to take and apply samples to the panel, and descriptions of biomarker levels and their meaning. A method of detecting the presence of disease, by taking a sample of an individual, applying the sample to the diagnostic panel including at least one biomarker indicative of disease, detecting the presence of at least one biomarker, comparing levels of the biomarker to a baseline, and determining if the individual has a disease. Methods of determining the stage of a disease, monitoring the progress of disease treatments, determining viral suppression or rebound, and detecting latent virus.

### BACKGROUND OF THE INVENTION

### 1. TECHNICAL FIELD

The present invention relates to methods and diagnostics for determining disease states in individuals infected by viruses based on biomarker profiles. More specifically, the present invention relates to methods and diagnostics for evaluating gene editor treatment of viruses.

### 2. BACKGROUND ART

Many individuals suffer from viral infections, such as HIV and hepatitis, which replicate inside the cells of an individual. Viruses can be latent within the cell, meaning that they are inactive and do not produce symptoms of disease but can become active at a later time. Antiviral drugs are routinely given to those suffering from viruses, but antivirals cannot address the problem of latent viruses. Therefore, it can be difficult to determine whether an individual is free from virus after treatment.

PCT/US2013/068592 to Slupsky discloses a method of determining the state of a disease in a subject by obtaining first and second sets of biological samples from subjects known to have first and second states of the disease, each biological sample comprising a plurality of biomarkers, the first and second states being differentiated by a predetermined period of time; for each sample, generating a profile for each state of the disease based on concentrations of biomarkers from a plurality of samples; generating a profile based on a biological sample from a subject having an unknown state of disease; and comparing the profile for the unknown state of disease to the profiles of the first and second states of disease to determine whether the subject has one of the first state of disease or the second state of disease. This method is used to determine whether an individual has HIV and was infected recently.

There remains a need for a method of determine the state of infection of any type of virus, as well as whether therapies are working to treat the virus.

### SUMMARY OF THE INVENTION

The present invention provides for a diagnostic panel including a test for detecting at least one biomarker that indicates the presence of a virus.

The present invention provides for a kit including the diagnostic panel, instructions for use, materials to take and apply samples to the panel, and descriptions of biomarker levels and their meaning.

The present invention provides for a method of detecting the presence of disease, by taking a sample of an individual, applying the sample to the diagnostic panel including at least one biomarker indicative of disease, detecting the presence of at least one biomarker, comparing levels of the biomarker to a baseline, and determining if the individual has a disease.

The present invention further provides for a method of determining the stage of a disease, by taking a sample of an individual, applying the sample to the diagnostic panel including at least one biomarker indicative of disease, detecting the presence of at least one biomarker, comparing levels of the biomarker to known stage levels, and determining the stage of the disease in the individual.

The present invention also provides for a method of monitoring the progress of disease treatments, by taking a sample of an individual, applying the sample to the diagnostic panel including at least one biomarker indicative of disease, detecting the presence of at least one biomarker, comparing levels of the biomarker to a baseline, and determining if the treatment is working to reverse or prevent the disease.

The present invention provides for a method of determining viral suppression or rebound by taking a sample of an individual receiving treatment of gene editing therapeutics, antiviral treatment, or combinations thereof, applying the sample to the diagnostic panel including at least one biomarker indicative of disease, detecting the presence of at least one biomarker, comparing levels of the biomarker to a baseline, and determining if latent virus has been activated.

The present invention provides fora method of detecting latent virus by taking a sample of an individual, applying the sample to the diagnostic panel including at least one biomarker indicative of disease, detecting the presence of at least one biomarker, comparing levels of the biomarker to a baseline, and determining if latent virus is present in the individual.

### DETAILED DESCRIPTION OF THE INVENTION

The present invention is generally directed to a diagnostic panel and method for determining the presence of a virus by determining the level of at least one metabolite or biomarker, and determining the metabolic state of an individual. The diagnostic panel can determine disease state and viral suppression or rebound during various treatments.

The term “assay” as used herein refers to a procedure that determines the amount of a particular constituent of a mixture or sample. “Assay” can interchangeably be used with the term “test” herein.

The term “biomarker” as used herein refers to a substance, such as, but not limited to, a protein, DNA sequence, RNA sequence, metabolite, or other biological substance or substances that, when detected, indicates a particular healthy or unhealthy state of an individual with respect to disease, and especially viruses in an activated and/or inactive state.

The term “healthy” as used herein refers to a state of an individual who is free from disease, is in good health, and has relatively low risk of developing disease.

The term “sample” as used herein refers to a biological sample from an individual, and can be, but is not limited to, blood, plasma, urine, saliva, tears, tissue, or cerebral spinal fluid (CSF).

The term “stage of disease” as used herein refers to the progression of disease in an individual. The stage can be aggressive, active, acute, recent, chronic, indolent, non-recent, primary, persistent, remission or subclinical. The stage can also be the absence of disease.

The term “individual” as used herein refers preferably to a human, but can also refer to any animal, such as, but not limited to, a monkey, dog, cat, rabbit, bat, horse, sheep, cow, pig, mouse, or rat suffering from disease.

Most generally, the diagnostic panel includes of a set of chemical, immunochemical and/or enzymatic assays or tests that can be used together for monitoring the levels of a set of biomarkers. The diagnostic panel can be used to determine the presence of disease, or the propensity of an individual to develop disease. The diagnostic panel can also be used to mark the progression of disease and disease stages, such as a recent infection (contracted less than 6 months ago) or chronic infection (contracted over 12 months ago). Various references or baselines can be created for each stage of disease that can include likely levels of particular biomarkers for that stage of disease. Evaluation of different stages or components of disease is important for intervention or reversal of the effects of the disease. The diagnostic panel can be used to confirm eradication of the disease after treatment. Most preferably, the diagnostic panel includes a test for detecting at least one biomarker that indicates the presence of a virus.

The biomarkers are preferably metabolites that are indicative of the presence of a disease, and especially a virus. Metabolites are those chemicals (generally less than 1,000 Da) that are involved in cellular reactions for energy production, growth, development, signaling and reproduction, and can be taken up, or released from cells according to cellular needs. These chemicals include sugars, amino acids, organic acids, as well as xenobiotic compounds. Metabolomics (or metabonomics as it is sometimes referred) is dedicated to the study of all metabolites in a cell or system and changes that might result from an internal or external stress such as an infection, disease state, or exposure to a toxin. Metabolic changes can result from changes in the chemical reactions that use these metabolites (i.e. metabolic pathways), or the transporters that take up or release these metabolites. Infection of a person by a virus or bacterium causes major changes both at the cellular level (the site of infection), and systemically (through the innate immune response). These responses include, but are not limited to, signaling of specific immune cells, signaling of apoptosis, changes in transporters, as well as changes in mitochondrial function and energy production—changes that can be observed as changes in metabolite concentrations at the cellular level, and systemically in the blood or urine.

The metabolites can include, but are not limited to, 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5 (which may be methylamine), metabolite 6 (which may be methylguanidine), N,N-dimethylglycine, O-acetylcarnitine, pantothenate, propylene glycol, pyroglutamate, pyruvate, quinolinate, serine, succinate, sucrose, metabolite 7 (which may be tartrate), taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, and combinations thereof.

The metabolite can also be any from the following metabolic cycles:

Polypurine: guanosine, guanine, xanthine, uric acid, adenosine, inosine, inosinic acid, hypoxanthine, xanthine, CO₂, H₂O, urea, N-carboamoyl-β-alanine, beta-alanine, ammonia, and (β-aminoisobutyrate.

Polyamines: putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, and FDP-lysine protein.

KREBS/TCA cycle: threo-Ds-isocitrate, oxalo-succinate, 2-oxo-glutarate, oxalo-acetate, L-glutamate, 2-hydroxy-glutarate, pyruvate, acetyl-CoA, cis-Aconitate, D-isocitrate, α-ketoglutarate, succinyl-CoA, succinate, fumarate, malate, glycine, citrate, carnitine, (−)O-acetyl-carnitine, cis-aconitate, itaconate, glycolate, glyoxylate, oxalate, oxalyl-CoA, formate, formyl-CoA, and CO₂.

Glycolysis and gluconeogenesis: glucose, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), pyruvate, D-glucose, D-glucono-1,5-lactone, D-gluconate, α-D-mannose 6-P, D-mannose, D-fructose, D-sorbitol, glycerone-P, sn-glycerol-3P, glycerol, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, and D-lactate.

Oxidative phosphorylation: adenosine triphosphate (ATP), adenosine diphosphate (ADP), H+, succinate, fumarate, H₂O, O₂, NADH, and NAD+.

Pentose phosphate: glucose-6-phosphate, NADP+, NADPH, 6-phosphogluconolatone, H₂O, H+, 6-phosphogluconate, CO₂, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, and xylulose 5-phosphate, D-ribulose, D-ribitol, D-ribose, L-ribulose, sedoheptulose 1,7P₂, and 3-oxo-6-P-hexulose.

Urea cycle: L-ornithine, carbamoyl phosphate, L-citrulline, argininosuccinate, fumarate, L-arginine, urea, L-aspartate, adenosine diphosphate (ADP), adenosine monophosphate (AMP), and pyrophosphate.

Fatty acid β-oxidation: trans-Δ²-enoyl-CoA, L-β-hydroxyacyl CoA, β-ketoacyl CoA, FADH2, NADH, acetyl-CoA, acyl-CoA, propionyl-CoA, and succinyl-CoA.

Nucleotide metabolism: AMP, inosine monophosphate (IMP), xanthosine monophosphate (XMP), guanosine monophosphate (GMP), ribose-5-phosphate, adenosine, inosine, hypoxanthine, xanthosine, xanthine, guanosine, guanine, uric acid, fumarate, adenylosuccinate, uridine, uridine monophosphate (UMP), ADP, thymidine, thymine, deoxyribose-1-phosphate, deoxythymidine monophosphate (dTMP), deoxycytidine, ATP, and deoxycytidine monophosphate (dCMP).

Cofactors and vitamins: retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NAD+, NADH, ADP, and ATP.

Amino acid metabolism: glutamate, NH4+, α-ketoglutarate, pyruvate, oxaloacetate, glutamate γ-semialdehyde, Δ¹-pyrroline-5-carboxylate, citrulline, arginine, urea, ornithine, glycine, CO₂, NH₃, N⁵,N¹⁰-methyleneTHF, 3-phosphoglycerate, α-ketobutyrate, propionyl-CoA, succinyl-CoA, acetyl-CoA, serine, α-amino-β-ketobutyrate, aminoacetone, cysteine sulfinate, β-sulfinylpyruvate, bisulfite, sulfite, sulfate, alanine, glutathione, taurine, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, tyrosine, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, carnitine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, quinolinate, glutaryl-CoA, and acetoacetyl-CoA.

A single metabolite can be used, as well as any combination of metabolites in determining disease state.

Preferably, the disease detected with the diagnostic panel is a virus, such as, but not limited to, human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, or yellow fever virus.

In general, the diagnostic panel can use a support structure such as a flat microwell plate (such as an ELISA plate) that has multiple wells to hold samples. Various enzymes or antibodies can be applied to the wells as needed for each test. A housing can enclose the diagnostic panel to prevent contamination or unwanted spread of samples, in plastic or another suitable material. Various sensors can be included to sense concentration data of biomarkers. A processor can analyze the concentration data and provide results of the presence of disease or particular disease stage.

Various methods can be used to detect the presence of the biomarkers, such as, but not limited to, liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, or infrared spectroscopy.

The diagnostic panel of the present invention can be included in a kit. The kit can include the diagnostic panel, instructions for use, materials to take and apply samples to the panel (such as, but not limited to, swabs, syringes, or vials), and descriptions of biomarker levels and their meaning (such as normal values). The kit can include various antibodies as needed to detect the biomarkers.

The present invention provides for a method of detecting the presence of disease, by taking a sample of an individual, applying the sample to the diagnostic panel including at least one biomarker indicative of disease, detecting the presence of at least one biomarker, comparing levels of the biomarker to a baseline, and determining if the individual has a disease. Most preferably, the biomarker is indicative of the presence of a virus, such as any described above. The biomarkers can be detected by any of the methods described above. Baselines for the presence of disease can be created based on individuals known to have a disease. Different concentrations of the biomarkers can indicate the presence of disease. If the individual is determined to have a disease, treatment can be recommended.

The present invention further provides for a method of determining the stage of a disease, by taking a sample of an individual, applying the sample to the diagnostic panel including at least one biomarker indicative of disease, detecting the presence of at least one biomarker, comparing levels of the biomarker to known stage levels, and determining the stage of the disease in the individual. The biomarkers can be detected by any of the methods described above. Various stage level baselines can be created by methods known in the art based on individuals known to have a particular stage. Different concentrations of the biomarkers can indicate a different stage. Depending on the stage determined, treatment can be recommended to the individual.

The present invention also provides for a method of monitoring the progress of disease treatments, by taking a sample of an individual, applying the sample to the diagnostic panel including at least one biomarker indicative of disease, detecting the presence of at least one biomarker, comparing levels of the biomarker to a baseline, and determining if the treatment is working to reverse or prevent the disease. The biomarkers can be detected by any of the methods described above. Baselines can be created as described above.

The treatment can be a gene editing therapeutic, such as, but not limited to CRISPR Cas9, CRISPR Cfp1, ZFNs, TALENS, Albumin-based editors, and other CRISPR-associated nucleases such as C2c1, C2c3, TevCas9, Archaea Cas9, CasY.1-CasY.6, CasX gRNAs, or Argonaute endonuclease gDNAs. The treatment can also be any other antiviral treatment (protease inhibitors, integrase inhibitors, RT inhibitors) such as, but not limited to, abacavir, aciclovir, acyclovir, adefovir, amantadine, amprenavir, ampligen, arbidol, atazanavir, atripla, balavir, cidofovir, combivir, dolutegravir, darunavir, delaviridine, didanosine, docosanol, edoxudine, efavirenz, emtricitabine, enfuvirtide, entecavir, ecoliever, famciclovir, fomivirsen, fosamprenavir, foscarnet, fosfonet, fusion inhibitor, ganciclovir, ibacitabine, imunovir, idoxuridine, imiquimod, indinavir, inosine, interferon, interferon type I, interferon type II, interferon type III, lamivudine, lopinavir, loviride, maraviroc, moroxydine, methisazone, nelfinavir, nevirapine, nexavir, nitazoxanide, nucleoside analogues, novir, oseltamivir, peginterferon α-2a, penciclovir, peramivir, pleconaril, podophyllotoxin, raltegravir, ribavirin, rimantadine, ritonavir, pyramidine, saquinavir, sofosbuvir, telaprevir, tenofovir, tenofovir disoproxil, tipranavir, trifuridine, trizivir, tromantadine, truvada, valaciclovir, valganciclovir, vicriviroc, vidarabine, viramidine, zalcitabine, zanamivir, or zidovudine. The treatment can also be a combination of a gene editing therapeutic and antiviral treatment. Based on the results of the biomarker levels, the treatment prescribed or the dose can be adjusted if necessary to improve its effect in the individual.

The diagnostic panel can be used to determine viral suppression or rebound during treatment of an individual with either gene editing therapeutics or antiviral treatment, or with a combination treatment. In this manner, latent populations of a virus can be monitored for activation, and treatment can be adjusted if active virus is found. A different treatment can be prescribed, or the dose of the current treatment can be altered based on the results. For example, after initial infection with the HIV virus, the primary HIV infection subsides within a few weeks to a few months, and is typically followed by a long clinical “latent” period which may last for up to 10 years. The latent period is also referred to as asymptomatic HIV infection or chronic HIV infection. The subject's CD4 lymphocyte numbers rebound, but not to pre-infection levels and most subjects undergo seroconversion, that is, they have detectable levels of anti-HIV antibody in their blood, within 2 to 4 weeks of infection. During this latent period, there can be no detectable viral replication in peripheral blood mononuclear cells and little or no culturable virus in peripheral blood. During the latent period, also referred to as the clinical latency stage, people who are infected with HIV may experience no HIV-related symptoms, or only mild ones. At a later time, the virus can become activated and the individual can experience symptoms again. The present invention can be used to detect when this activation has begun so that appropriate treatment can be prescribed to the individual. Therefore, the present invention provides for a method of determining viral suppression or rebound by taking a sample of an individual receiving treatment of gene editing therapeutics, antiviral treatment, or combinations thereof, applying the sample to the diagnostic panel including at least one biomarker indicative of disease, detecting the presence of at least one biomarker, comparing levels of the biomarker to a baseline, and determining if latent virus has been activated.

A large percent of latent virus still produces basal level amounts of viral protein in an inactivated state. Basal protein will still cause a biochemical shift that is indirectly detectable by measuring metabolite shifting. Therefore, it can be determined whether there is latent virus present in an individual by comparing results from the diagnostic panel to a baseline wherein no virus is present. This can be useful to detect a virus before the individual has started to present any symptoms, if the individual has stopped presenting any symptoms, determining if the individual is at risk of developing disease at a later time, or if they should avoid certain therapeutics. Therefore, the present invention provides for a method of detecting latent virus by taking a sample of an individual, applying the sample to the diagnostic panel including at least one biomarker indicative of disease, detecting the presence of at least one biomarker, comparing levels of the biomarker to a baseline, and determining if latent virus is present in the individual.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A diagnostic panel comprising a test for detecting at least one biomarker that indicates the presence of a virus.

2. The diagnostic panel of claim 1, wherein said at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, a-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, γ-ketobutyrate, γ-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

3. The diagnostic panel of claim 1, wherein said virus is chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

4. The diagnostic panel of claim 1, wherein said panel includes a support structure of a flat microwell plate.

5. The diagnostic panel of claim 1, wherein said panel is enclosed in a housing.

6. The diagnostic panel of claim 1, wherein said panel further includes sensors that sense a concentration of said at least one biomarker.

7. A kit comprising a diagnostic panel including a test for detecting at least one biomarker that indicates the presence of a virus, instructions for use, materials to take and apply samples to said panel, and descriptions of biomarker levels and their meaning.

8. The kit of claim 7, wherein said at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

9. A method of detecting the presence of disease, including the steps of:
taking a sample of an individual;
applying the sample to a diagnostic panel including at least one biomarker indicative of disease;
detecting the presence of at least one biomarker;
comparing levels of the biomarker to a baseline; and
determining if the individual has a disease.

10. The method of claim 9, wherein the at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H2O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-ΔA²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

11. The method of claim 9, wherein the disease is a virus chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

12. The method of claim 9, wherein said detecting step includes using a method chosen from the group consisting of liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, and infrared spectroscopy.

13. The method of claim 9, wherein the sample is chosen from the group consisting of blood, plasma, urine, saliva, tears, tissue, and cerebral spinal fluid (CSF).

14. A method of determining the stage of a disease, including the steps of:
taking a sample of an individual;
applying the sample to a diagnostic panel including at least one biomarker indicative of disease;
detecting the presence of at least one biomarker;
comparing levels of the biomarker to known stage levels; and
determining the stage of the disease in the individual.

15. The method of claim 14, wherein the at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

16. The method of claim 14, wherein the disease is a virus chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

17. The method of claim 14, wherein said detecting step includes using a method chosen from the group consisting of liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, and infrared spectroscopy.

18. The method of claim 14, wherein the sample is chosen from the group consisting of blood, plasma, urine, saliva, tears, tissue, and cerebral spinal fluid (CSF).

19. A method of monitoring the progress of disease treatments, including the steps of:
taking a sample of an individual;
applying the sample to a diagnostic panel including at least one biomarker indicative of disease;
detecting the presence of at least one biomarker;
comparing levels of the biomarker to a baseline; and
determining if the treatment is working to reverse or prevent the disease.

20. The method of claim 19, wherein the at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, α-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

21. The method of claim 19, wherein the disease being treated is a virus chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

22. The method of claim 19, wherein said detecting step includes using a method chosen from the group consisting of liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, and infrared spectroscopy.

23. The method of claim 19, wherein the sample is chosen from the group consisting of blood, plasma, urine, saliva, tears, tissue, and cerebral spinal fluid (CSF).

24. The method of claim 19, wherein the treatment is chosen from the group consisting of CRISPR Cas9, CRISPR Cfp1, ZFNs, TALENS, Albumin-based editors, C2c1, C2c3, TevCas9, Archaea Cas9, CasY.1-CasY.6, CasX gRNAs, Argonaute endonuclease gDNAs, abacavir, aciclovir, acyclovir, adefovir, amantadine, amprenavir, ampligen, arbidol, atazanavir, atripla, balavir, cidofovir, combivir, dolutegravir, darunavir, delaviridine, didanosine, docosanol, edoxudine, efavirenz, emtricitabine, enfuvirtide, entecavir, ecoliever, famciclovir, fomivirsen, fosamprenavir, foscarnet, fosfonet, fusion inhibitor, ganciclovir, ibacitabine, imunovir, idoxuridine, imiquimod, indinavir, inosine, interferon, interferon type I, interferon type II, interferon type III, lamivudine, lopinavir, loviride, maraviroc, moroxydine, methisazone, nelfinavir, nevirapine, nexavir, nitazoxanide, nucleoside analogues, novir, oseltamivir, peginterferon α-2a, penciclovir, peramivir, pleconaril, podophyllotoxin, raltegravir, ribavirin, rimantadine, ritonavir, pyramidine, saquinavir, sofosbuvir, telaprevir, tenofovir, tenofovir disoproxil, tipranavir, trifuridine, trizivir, tromantadine, truvada, valaciclovir, valganciclovir, vicriviroc, vidarabine, viramidine, zalcitabine, zanamivir, zidovudine, and combinations thereof.

25. The method of claim 19, further including the step of adjusting the treatment based on biomarker levels to improve effects in the individual.

26. A method of determining viral suppression or rebound, including the steps of:
taking a sample of an individual having a virus receiving treatment chosen from the group consisting of gene editing therapeutics, antiviral treatment, or combinations thereof;
applying the sample to a diagnostic panel including at least one biomarker indicative of disease;
detecting the presence of at least one biomarker;
comparing levels of the biomarker to a baseline; and
determining if latent virus has been activated.

27. The method of claim 26, wherein the at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, a-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, γ-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

28. The method of claim 26, wherein the virus is chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

29. The method of claim 26, wherein said detecting step includes using a method chosen from the group consisting of liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, and infrared spectroscopy.

30. The method of claim 26, wherein the sample is chosen from the group consisting of blood, plasma, urine, saliva, tears, tissue, and cerebral spinal fluid (CSF).

31. The method of claim 26, wherein the gene editing therapeutics are chosen from the group consisting of CRISPR Cas9, CRISPR Cfp1, ZFNs, TALENS, Albumin-based editors, C2c1, C2c3, TevCas9, Archaea Cas9, CasY.1-CasY.6, CasX gRNAs, or Argonaute endonuclease gDNAs, and combinations thereof.

32. The method of claim 26, wherein the antiviral therapeutics are chosen from the group consisting of abacavir, aciclovir, acyclovir, adefovir, amantadine, amprenavir, ampligen, arbidol, atazanavir, atripla, balavir, cidofovir, combivir, dolutegravir, darunavir, delaviridine, didanosine, docosanol, edoxudine, efavirenz, emtricitabine, enfuvirtide, entecavir, ecoliever, famciclovir, fomivirsen, fosamprenavir, foscarnet, fosfonet, fusion inhibitor, ganciclovir, ibacitabine, imunovir, idoxuridine, imiquimod, indinavir, inosine, interferon, interferon type I, interferon type II, interferon type III, lamivudine, lopinavir, loviride, maraviroc, moroxydine, methisazone, nelfinavir, nevirapine, nexavir, nitazoxanide, nucleoside analogues, novir, oseltamivir, peginterferon α-2a, penciclovir, peramivir, pleconaril, podophyllotoxin, raltegravir, ribavirin, rimantadine, ritonavir, pyramidine, saquinavir, sofosbuvir, telaprevir, tenofovir, tenofovir disoproxil, tipranavir, trifuridine, trizivir, tromantadine, truvada, valaciclovir, valganciclovir, vicriviroc, vidarabine, viramidine, zalcitabine, zanamivir, zidovudine, and combinations thereof.

33. The method of claim 26, further including, if latent virus is activated, the step of treating the individual for the latent virus.

34. A method of detecting latent virus, including the steps of:
taking a sample of an individual;
applying the sample to a diagnostic panel including at least one biomarker indicative of disease;
detecting the presence of at least one biomarker;
comparing levels of the biomarker to a baseline; and
determining if latent virus is present in the individual.

35. The method of claim 34, wherein the at least one biomarker is a metabolite chosen from the group consisting of 1,3-dimethylurate, levoglucosan, 1-methylnicotinamide, metabolite 1,2-hydroxyisobutyrate, 2-oxoglutarate, 3-aminoisobutyrate, 3-hydroxybutyrate, 3-hydroxyisovalerate, 3-indoxylsulfate, 4-hydroxyphenylacetate, 4-hydroxyphenyllactate, 4-pyridoxate, acetate, acetoacetate, acetone, adipate, alanine, allantoin, asparagine, betaine, carnitine, citrate, creatine, creatinine, dimethylamine, ethanolamine, formate, fucose, fumarate, glucose, glutamine, glycine, metabolite 2, metabolite 3, hippurate, histidine, hypoxanthine, isoleucine, lactate, leucine, lysine, mannitol, metabolite 4, metabolite 5, metabolite 6, N,N-dimethylglycine, O-acetylcarnitine, pantothenate, propylene glycol, pyroglutamate, pyruvate, quinolinate, serine, succinate, sucrose, metabolite 7, taurine, threonine, trigonelline, trimethylamine-N-oxide, tryptophan, tyrosine, uracil, urea, valine, xylose, cis-aconitate, myo-inositol, trans-aconitate, 1-methylhistidine, 3-methylhistidine, ascorbate, phenylacetylglutamine, 4-hydroxyproline, gluconate, galactose, galactitol, galactonate, lactose, phenylalanine, proline betaine, trimethylamine, butyrate, propionate, isopropanol, mannose, 3-methylxanthine, ethanol, benzoate, glutamate, glycerol, guanosine, guanine, xanthine, uric acid, adenosine, inosine, inosinic acid, CO₂, H₂O, N-carboamoyl-β-alanine, beta-alanine, ammonia, (β-aminoisobutyrate, putrescine, spermidine, spermine, methionine, S-adenosylmethionine, decarboxylated S-adenosylmethionine, arginine, ornithine, putrescine, N1-acetylspermidine, N1-acetylspermine, elF5A(Lys), elF5A(Dhp), elF5A(Hpu), N1N2-diacetylspermine, 3-aminopropanal, 3-acetylaminopropanal, acrolein, FDP-lysine protein, threo-Ds-isocitrate, oxalo-succinate, 2-oxo-glutarate, oxalo-acetate, L-glutamate, 2-hydroxy-glutarate, acetyl-CoA, cis-aconitate, D-isocitrate, α-ketoglutarate, succinyl-CoA, malate, (−)O-acetyl-carnitine, itaconate, glycolate, glyoxylate, oxalate, oxalyl-CoA, formyl-CoA, glucose 6-phosphate (G6P), fructose 6-phosphate (F6P), fructose 1,6-biphosphate (F1,6BP), glyceraldehyde 3-phosphate (GADP), dihydroxyacetone phosphate (DHAP), 1,3-bisphosphoglyceric acid (1,3BPG), 3-phosphoglyceric acid (3PG), 2-phosphoglyceric acid (2PG), phosphoenolpyruvic acid (PEP), D-glucose, D-glucono-1,5-lactone, D-gluconate, a-D-mannose 6-P, D-mannose, D-fructose, D-sorbitol, glycerone-P, sn-glycerol-3P, D-glyceraldehyde, 1,2 propane-diol, 2-hydroxypropionaldehyde, 3-P-serine, 3-P-hydroxypyruvate, D-glycerate, hydroxypyruvate, L-alanine, L-alanyl-tRNA, L-glutamate, 2-oxoglutarate, L-lactate, D-lactate, adenosine triphosphate (ATP), adenosine diphosphate (ADP), H+, succinate, O₂, NADH, NAD+, NADP+, NADPH, 6-phosphogluconolatone, 6-phosphogluconate, ribulose-5-phosphate, ribose-5-phosphate, xylulose-5-phosphate, glyceraldehyde 3-phosphate, sedoheptulose 7-phosphate, fructose 6-phosphate, erythrose 4-phosphate, xylulose 5-phosphate, D-ribulose, D-ribitol, D-ribose, L-ribulose, sedoheptulose 1,7P₂, 3-oxo-6-P-hexulose, L-ornithine, carbamoyl phosphate, L-citrulline, argininosuccinate, L-arginine, L-aspartate, adenosine monophosphate (AMP), pyrophosphate, trans-Δ²-enoyl-CoA, L-β-hydroxyacyl CoA, β-ketoacyl CoA, FADH2, acyl-CoA, propionyl-CoA, inosine monophosphate (IMP), xanthosine monophosphate (XMP), guanosine monophosphate (GMP), xanthosine, adenylosuccinate, uridine, uridine monophosphate (UMP), thymidine, thymine, deoxyribose-1-phosphate, deoxythymidine monophosphate (dTMP), deoxycytidine, deoxycytidine monophosphate (dCMP), retinyl palmitate, palmitate, palmityl-CoA, retinoate, β-glucuronide, retinal, β-carotene, retinoic acid, calcidiol, 25-hydroyergocalciferol, calcitriol, methylcobalamin, 5′-deoxyadenosylcobalamin, α-CECH, NH₄+, α-ketoglutarate, oxaloacetate, glutamate γ-semialdehyde, Δ¹-pyrroline-5-carboxylate, citrulline, NH₃, N⁵,N¹⁰-methyleneTHF, 3-phosphoglycerate, α-ketobutyrate, α-amino-β-ketobutyrate, aminoacetone, cysteine sulfinate, β-sulfinylpyruvate, bisulfite, sulfite, sulfate, glutathione, hypotaurine, adenosine 5′-phosphosulfate, 3′-phosphoadenosine 5′-phosphosulfate, homocysteine, α-keto-β-methylvalerate, α-ketoisocaproate, α-ketoisovalerate, α-methylbutyryl-CoA, tiglyl-CoA, 3-methyl-3-hydroxybutyryl-CoA, 2-methylacetoacetyl-CoA, isovaleryl-CoA, 3-methylcrotonyl-CoA, 3-methylglutaconyl-CoA, 3-hydroxy-3-methylglutaryl-CoA, acetoacetate, isobutyryl CoA, methacrylyl-CoA, 3-hydroxyisobutyryl-CoA, methylmalonic semialdehyde, p-hydroxyphenylpyruvate, homogentisate, 4-maleylacetoacetate, 4-fumarylacetoacetate, fumarate, 3-hydroxytrimethyllysine, 4-N-trimethylaminobutyraldehyde, γ-butyrobetaine, urocanate, 4-imidazolone-5-propionate, N-formimidoyl-L-glutamate, N⁵-formimino-tetrahydrofolate, histamine, N-formyl-kynurenine, kynurenine, kynurenate, 3-hydroxykynurenine, anthranilate, 3-hydroxyanthranilate, glutaryl-CoA, acetoacetyl-CoA, and combinations thereof.

36. The method of claim 34, wherein the disease is a virus chosen from the group consisting of human immunodeficiency virus (HIV), herpes simplex virus (HSV-1 and HSV-2), human T-lymohotropic virus (HTLV), John Cunningham virus (JC Virus), vesicular stomatitis virus (VSV), hepatitis C virus (HCV), hepatitis B virus (HBV), Zika virus, Dengue virus, Chikungunya virus, Ebola virus, adeno-associated virus, aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Cosavirus A, Cowpox virus, Coxsackievirus, Crimean-Congo hemorrhagic fever virus, Dhori virus, Dugbe virus, Duvenhage virus, Eastern equine encephalitis virus, Echovirus, Encephalomyocarditis virus, Epstein-Barr virus, European bat lyssavirus, Hepatitis G virus, Hantaan virus, Hendra virus, Hepatitis A virus, Hepatitis E virus, Hepatitis delta virus, Horsepox virus, human adenovirus, human astrovirus, human coronavirus, human cytomegalovirus, human enterovirus 68, human enterovirus 70, human herpesvirus 6, human herpesvirus 7, human herpes virus 8, human papillomavirus (HPV) 1, HPV 2, HPV 16, HPV 18, human parainfluenza, human parvovirus B19, human respiratory syncytial virus, human rhinovirus, human severe acute respiratory syndrome (SARS) coronavirus, human spumaretrovirus, human torovirus, influenza A virus, influenza B virus, influenza C virus, Isfahan virus, JC polyomavirus, Japanese encephalitis virus, Junin arenavirus, KI polyomavirus, Kunjin virus, Lagos bat virus, Lake Victoria marburgvirus, Langat virus, Lassa virus, Lordsdale virus, Louping ill virus, lymphocytic choriomeningitis virus, Machupo virus, Mayaro virus, Middle East Respiratory Syndrome (MERS) coronavirus, measles virus, Mengo encephalomyocarditis virus, Merkel cell polyomavirus, Mokola virus, Molluscum contagiousum virus, monkeypox virus, mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, sandfly fever Sicilian virus, Sapporo virus, Semliki forest virus, Seoul virus, simian foamy virus, simian virus 5, Sindbis virus, Southhampton virus, St. Louis encephalitis virus, tick-borne powassan virus, torque teno virus, Toscana virus, Ulukuniemi virus, vaccinia virus, varicella-zoster virus, variola virus, Venezuelan equine encephalitis virus, vesicular stomatitis virus, western equine encephalitis virus, WU polyomavirus, West Nile virus, Yaba monkey tumor virus, Yaba-like disease virus, and yellow fever virus.

37. The method of claim 34, wherein said detecting step includes using a method chosen from the group consisting of liquid chromatography, gas chromatography, liquid chromatography-mass spectrometry, gas chromatography-mass spectrometry, high performance liquid chromatography-mass spectrometry, capillary electrophoresis-mass spectrometry, nuclear magnetic resonance spectrometry (NMR), raman spectroscopy, and infrared spectroscopy.

38. The method of claim 34, wherein the sample is chosen from the group consisting of blood, plasma, urine, saliva, tears, tissue, and cerebral spinal fluid (CSF).
